(19) United States
(12) Patent Application Publication
(10) Pub. No.: US 2021/0393614 A1
(43) Pub. Date: Dec. 23, 2021

(54) QUINONE REDUCTASE 2 INHIBITORS FOR USE AS NEUROPROTECTIVE AGENTS

(71) Applicant: DUKE UNIVERSITY, Durham, NC (US)

(72) Inventors: Christopher D. Lascola, Durham, NC (US); Jesse Keiser Fitzpatrick, San Francisco, CA (US)

(21) Appl. No.: 17/285,581

(22) PCT Filed: Oct. 16, 2019
(86) PCT No.: PCT/US2019/056530

§ 371 Date: Apr. 15, 2021

Related U.S. Application Data
(60) Provisional application No. 62/746,868, filed on Oct. 17, 2018.

(51) Int. Cl. A61K 31/4706; A61K 31/4709; A61P 25/28

(52) U.S. Cl. CPC A61K 31/4706; A61P 25/28; A61K 31/4709

(57) ABSTRACT

Provided herein according to some embodiments is a method of treating acute neural injury in a subject in need thereof, comprising administering to the subject a compound of Formula I or Formula II. Also provided is a method of treating vascular dementia in a subject in need thereof, comprising administering to the subject a compound of Formula I or Formula II. Further provided is a method of treating CNS lupus in a subject in need thereof, comprising administering to the subject a compound of Formula I or Formula II.

### BACKGROUND

Aminoquinolines, with chloroquine (CQ) and hydroxychloroquine (HQ) as prototypes, are quinone reductase 2 (QR2) inhibitors that were originally developed to treat malaria but were subsequently found to have therapeutic efficacy for other indications, including, inter alia, autoimmune diseases such as systemic lupus erythematosis (SLE) and rheumatoid arthritis (RA). Singer et al., “Update on immunosuppressive therapy,” Curr. Opin. Rheumatol. 1998, 10:169-173; Wallace, “The use of chloroquine and hydroxychloroquine for non-infectious conditions other than rheumatoid arthritis or lupus: a crucial review,” Lupus 1996, 5 Suppl 1:S59-64. In SLE and RA, aminoquinolines are a mainstay of first-line therapy and are often used in combination with other medications. Aminoquinolines not only improve the signs and symptoms of SLE and RA but also have beneficial effects on lipid metabolism and reduce the occurrence of thrombosis. In patients with inflammatory or erosive osteoarthritis, similar benefits are observed. Efficacy has also been shown when used as adjunctive therapy in graft-vs-host disease, cancer, and HIV. Savarino et al., “Effects of chloroquine on viral infections: an old drug against today's diseases?” Lancet Infect. Dis. 2003, 3(11):722-7; Savarino et al., “Risks and benefits of chloroquine use in anticancer strategies,” Lancet Oncol. 2006, 7(10):792-3; Sotelo et al., “Adding chloroquine to conventional treatment for glioblastoma multiforme: a randomized, double-blind, placebo-controlled trial,” Ann. Intern. Med. 2006, 144(5):337-43.

The potential for chloroquine (CQ) in neuroprotection has been studied previously in preclinical models of stroke, excitotoxic and traumatic injuries, although the therapeutic mechanisms have remained elusive. CQ dramatically limits microglial and PMN migration into injury sites in the brain, decreases reactive astrogliosis and neovascularization, and reduces stroke volumes by 60% in a permanent MCA occlusion model. Giulian et al., “The role of mononuclear phagocytes in wound healing after traumatic injury to adult mammalian brain,” J. Neurosci. 1989, 9:4416-4429; Ivanova et al., “Cerebral ischemia enhances polyamine oxidation: identification of enzymatically formed 3-aminopropanal as an endogenous mediator of neuronal and glial cell death,” J. Exp. Med. 1998, 188:327-340. CQ also decreases cytokine production by microglial cells in vitro in response to various irritants. Giulian, “Microglia and the immune pathology of Alzheimer disease,” Am. J. Hum. Genet. 1999, 65:13-18.

Because some malaria is resistant to CQ, derivative compounds have also been explored. For example, US 2006/0074105 to Ware et al. describes certain quinoline and quinazoline derivatives said to be useful in the treatment of malaria and autoimmune diseases.

Though CQ and HQ are often used clinically as a first-line therapy in autoimmune disorders, their efficacy is limited by serious side effects. The most important and best-characterized toxicity is retinal, where long-term use may lead to “bull's eye maculopathy” and blindness unless dosing is limited. Cardiac toxicity, although rare, may also occur, manifesting either as conduction disturbances (e.g., bundle-branch block) and/or cardiomyopathy in association with congestive heart failure. Electron microscopy of cardiac and retinal biopsies after long-term CQ or HQ therapy reveals pathognomonic cytoplasmic inclusion bodies, understood to be a direct consequence of high drug accumulation in lysosomes (and melanosomes in retina and skin). Remarkably, CQ is capable of accumulating to mM concentration in skin, retinal, renal, and liver cells during therapeutic dosing while plasma concentrations remain <1 μM.

There remains a need to develop additional aminoquinoline quinone reductase 2 (QR2) inhibitors, particularly that also have diminished lysosomal accumulation in order to reduce toxicity.

### SUMMARY

Provided herein according to some embodiments is a method of treating acute neural injury in a subject in need thereof, comprising administering to the subject a compound of Formula I:

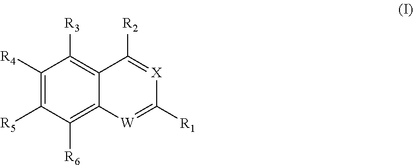

wherein:

W is N or N⁺O⁻;

X is CR₁₄ or N;

R₁ is H or trifluoromethyl;

R₂ is NR₇R₈, OR₁₁, SR₁₂, or alkyl;

R₃ is H or OR₁₃;

R₄ is H or methoxy;

R₅ is H, Cl, or trifluoromethyl;

R₆ is H, NR₉R₁₀ or trifluoromethyl;

R₇ is H, C₁₋₅ alkyl, heteroarylalkyl, cycloalkyl, heterocycloalkyl, heterocyclo, aryl, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkylhalo;

R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;

R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;

R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;

R₁₁ is alkyl, aryl or heteroaryl optionally substituted with alkyl, haloalkyl, aryl or heteroaryl;

R₁₂ is alkyl, aryl or heteroaryl optionally substituted with alkyl, haloalkyl, aryl or heteroaryl;

R₁₃ is alkyl or aryl optionally substituted with alkyl or haloalkyl; and

R₁₄ is H or aryl;

or a pharmaceutically acceptable salt or prodrug thereof.

In some embodiments, W is N. In some embodiments, X is CR₁₄. In some embodiments, R₁ is H. In some embodiments, R₂ is NR₇R₈. In some embodiments, R₃ is H. In some embodiments, R₄ is H. In some embodiments, R₅ is Cl. In some embodiments, R₆ is H. In some embodiments, R₇ is H. In some embodiments, R₈ is C₁₋₅ alkyl substituted with heteroaryl. In some embodiments, R₁₄ is H.

In some embodiments, the compound has a positive log D value at approximately pH 4 to pH 5.

In some embodiments, the compound is a compound of Formula I(a):

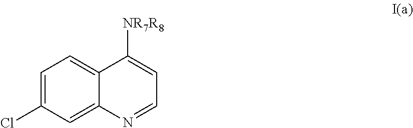

wherein R₇ and R₈ are each independently H or C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkylhalo).

In some embodiments, one of R₇ and R₈ is hydrogen, and the other is C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments, the compound has a positive log D value at approximately pH 4 to pH 5.

Also provided is a method of treating acute neural injury in a subject in need thereof, comprising administering to the subject a compound of Formula II:

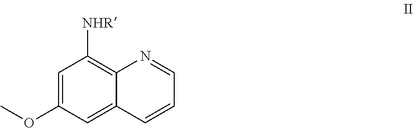

wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,

or a pharmaceutically acceptable salt or prodrug thereof.

In some embodiments, the compound has a positive log D value at approximately pH 4 to pH 5.

In some embodiments, the acute neural injury comprises traumatic brain injury. In some embodiments, the acute neural injury comprises subarachnoid hemorrhage. In some embodiments, the acute neural injury comprises post-operative cognitive deficit. In some embodiments, the acute neural injury comprises hypoxic brain injury. In some embodiments, the acute neural injury comprises ischemic brain injury.

Also provided is an active compound as taught herein for use in a method of treatment for an acute neural injury. Further provided is the use of an active compound as taught herein for the preparation of a medicament for the treatment of an acute neural injury. In some embodiments, the acute neural injury comprises traumatic brain injury. In some embodiments, the acute neural injury comprises subarachnoid hemorrhage. In some embodiments, the acute neural injury comprises post-operative cognitive deficit. In some embodiments, the acute neural injury comprises hypoxic brain injury. In some embodiments, the acute neural injury comprises ischemic brain injury.

Further provided is a method of treating vascular dementia in a subject in need thereof, comprising administering to the subject a compound of Formula I:

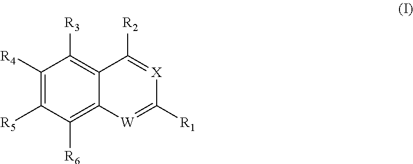

wherein:

W is N or N⁺O⁻;

X is CR₁₄ or N;

R₁ is H or trifluoromethyl;

R₂ is NR₇R₈, OR₁₁, SR₁₂, or alkyl;

R₃ is H or OR₁₃;

R₄ is H or methoxy;

R₅ is H, Cl, or trifluoromethyl;

R₆ is H, NR₉R₁₀ or trifluoromethyl;

R₇ is H, C₁₋₅ alkyl, heteroarylalkyl, cycloalkyl, heterocycloalkyl, aryl, heterocyclo, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkylhalo;

R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;

R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;

R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;

R₁₁ is alkyl, aryl or heteroaryl optionally substituted with alkyl, haloalkyl, aryl or heteroaryl;

R₁₂ is alkyl, aryl or heteroaryl optionally substituted with alkyl, haloalkyl, aryl or heteroaryl;

R₁₃ is alkyl or aryl optionally substituted with alkyl or haloalkyl; and

R₁₄ is H or aryl;

or a pharmaceutically acceptable salt or prodrug thereof.

In some embodiments, W is N. In some embodiments, X is CR₁₄. In some embodiments, R₁ is H. In some embodiments, R₂ is NR₇R₈. In some embodiments, R₃ is H. In some embodiments, R₄ is H. In some embodiments, R₅ is Cl. In some embodiments, R₆ is H. In some embodiments, R₇ is H. In some embodiments, R₈ is C₁₋₅ alkyl substituted with heteroaryl. In some embodiments, R₁₄ is H.

In some embodiments, the compound has a positive log D value at approximately pH 4 to pH 5.

In some embodiments, the compound is a compound of Formula I(a):

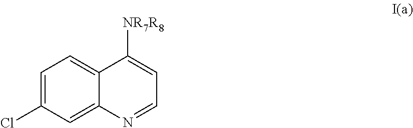

wherein R₇ and R₈ are each independently H or C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments, one of R₇ and R₈ is hydrogen, and the other is C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments, the compound has a positive log D value at approximately pH 4 to pH 5.

Also provided is a method of treating vascular dementia in a subject in need thereof, comprising administering to the subject a compound of Formula II:

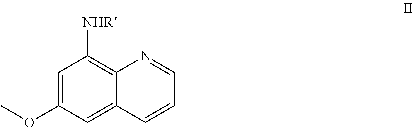

wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,
or a pharmaceutically acceptable salt or prodrug thereof.

In some embodiments, the compound has a positive log D value at approximately pH 4 to pH 5.

Also provided is an active compound as taught herein for use in a method of treatment for vascular dementia. Further provided is the use of an active compound as taught herein for the preparation of a medicament for the treatment of vascular dementia.

Still further provided is a method of treating CNS lupus in a subject in need thereof, comprising administering to the subject a compound of Formula I:

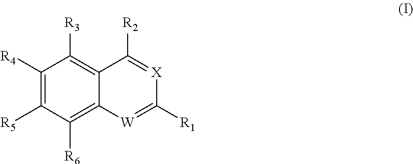

wherein:

W is N or N⁺O⁻;

X is CR₁₄ or N;

R₁ is H or trifluoromethyl;

R₂ is NR₇R₈, OR₁₁, SR₁₂, or alkyl;

R₃ is H or OR₁₃;

R₄ is H or methoxy;

R₅ is H, Cl, or trifluoromethyl;

R₆ is H, NR₉R₁₀ or trifluoromethyl;

R₇ is H, C₁₋₅ alkyl, heteroarylalkyl, cycloalkyl, heterocycloalkyl, heterocyclo, aryl, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkylhalo;

R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;

R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;

R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;

R₁₁ is alkyl, aryl or heteroaryl optionally substituted with alkyl, haloalkyl, aryl or heteroaryl;

R₁₂ is alkyl, aryl or heteroaryl optionally substituted with alkyl, haloalkyl, aryl or heteroaryl;

R₁₃ is alkyl or aryl optionally substituted with alkyl or haloalkyl; and

R₁₄ is H or aryl;

or a pharmaceutically acceptable salt or prodrug thereof.

In some embodiments, W is N. In some embodiments, X is CR₁₄. In some embodiments, R₁ is H. In some embodiments, R₂ is NR₇R₈. In some embodiments, R₃ is H. In some embodiments, R₄ is H. In some embodiments, R₆ is H. In some embodiments, R₇ is H. In some embodiments, R₈ is C₁₋₅ alkyl substituted with heteroaryl. In some embodiments, R₁₄ is H.

In some embodiments, the compound has a positive log D value at approximately pH 4 to pH 5.

In some embodiments, the compound is a compound of Formula I(a):

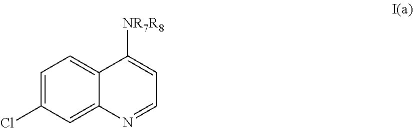

wherein R₇ and R₈ are each independently H or C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments, one of R₇ and R₈ is hydrogen, and the other is C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments, the compound has a positive log D value at approximately pH 4 to pH 5.

Also provided is a method of treating CNS lupus in a subject in need thereof, comprising administering to the subject a compound of Formula II:

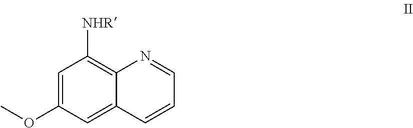

wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,

or a pharmaceutically acceptable salt or prodrug thereof.

In some embodiments, the compound has a positive log D value at approximately pH 4 to pH 5.

Also provided is an active compound as taught herein for use in a method of treatment for CNS lupus. Further provided is the use of an active compound as taught herein for the preparation of a medicament for the treatment of CNS lupus.

### BRIEF DESCRIPTION OF THE DRAWINGS

FIG. 1A-FIG. 1B: FIG. 1A shows a log D graph of chloroquine, and FIG. 1B shows a log D graph of hydroxychloroquine. The shaded regions in the figures represent the potential range of lysosomal pH encountered in vivo. At lysosomal pH, the log D of both chloroquine and hydroxychloroquine are substantially negative, reflecting the accumulated charge of these molecules and their loss of membrane permeability.

FIG. 2A-FIG. 2E show the log D graphs of Example Compounds A-E, respectively. Note that for each of the Example Compounds, log D values remain positive at lysosomal pH (between pH 4 and 5).

FIG. 3A-FIG. 3C: FIG. 3A provides diffusion weighted magnetic resonance imaging (DW-MRI) images of transient middle cerebral artery occlusion (MCAO) stroke evolution in mice at 4 hr (left) and 24 hr (right). FIG. 3B provides neurological scores (left) and rotorod assessment (right) with chloroquine (CQ) versus vehicle following MCAO. FIG. 3C shows cortical (left) and subcortical stroke volumes at three days, comparing Example Compound F (7C-4MAQ), chloroquine (CQ), QRII null mice, QRII null mice littermates, and vehicle.

FIG. 4 presents the results of rotorod (left) and Morris water maze (right) performance following TBI, comparing Example Compound F (7C-4MAQ) to chloroquine (CQ) and vehicle.

FIG. 5 shows T2 and susceptibility-weighted images (SWI) of hemorrhage in the right basal ganglia in the intracranial hemorrhage model.

FIG. 6 shows neuronal apoptosis after deep hypothermic circulatory arrest (DHCA). TUNEL analysis in cortex and hippocampus 48 hours after DHCA in rats treated with CQ (25 mg/kg, horizontal shaded bar), PBS (equal volume, black bar), QR2 inhibitor 7C-4MAQ (25 mg/kg, vertical shaded bar) or vehicle (50% DMSO, open bar).

FIG. 7 shows neuronal necrosis after DHCA. Acid fuchsin-celestin blue staining in cortex and hippocampus 48 hours after DHCA in rats treated with CQ (25 mg/kg, horizontal shaded bar), PBS (equal volume, black bar), QR2 inhibitor 7C-4MAQ (25 mg/kg, vertical shaded bar) or vehicle (50% DMSO, open bar).

FIG. 8 shows neurological outcome analyzed by neuroscore on postoperative days (POD) 1 and 2 in rats treated with QR2 inhibitor 7C-4MAQ (25 mg/kg, open bar) or 50% DMSO (solid bar) 2 hours before CPOB/DHCA.

FIG. 9 shows global brain perfusion as measured by MRI ADC-perfusion. A, ADC-perfusion intensity of sham mice compared to a pooled group of all mice with BCAS at day three and 32. 100% perfusion was defined as average ADC-perfusion intensity of sham group. Perfusion of BCAS mice (n=17) was significantly less than sham mice (n=5) at day three (**p<0.01), but perfusion rebounded to normal levels by day 32. B, representative colorized ADC-perfusion MR sequence superimposed onto a greyscale coronal T2 weighted sequence three days following BCAS surgery. Note the increased perfusion (increased ADC-perfusion signal intensity) in the sham brain compared to other treatment groups. C, ADC-perfusion at day three and 32 by treatment group. Perfusion in the sham group (n=5) was significantly higher than all other groups on day three (p<0.05, group effect; Sham×N-MCQ, n=4, p<0.01; Sham×CQ, n=5, p<0.01; Sham×Vehicle, n=9, p<0.05). Values represent averages±SEM. *p<0.05, **p<0.01.

FIG. 10 shows learning performance on the Morris water maze (MWM). A, escape latency. Mice administered 7C-4MAQ (“N-MCQ”) (n=10) exhibited decreased escape latencies compared to vehicle controls (Vehicle, n=10; p<0.05, group effect; p<0.01, N-MCQ×Vehicle), and were indistinguishable from sham mice (Sham, n=14). After the 5th day of MWM testing, the submerged platform was made visible and all difference between groups disappeared. B, escape latency. Animals administered CQ (n=10) had a performance profile similar to their N-MCQ counterparts (p<0.05, group effect; p<0.001, CQ×Vehicle). C, probe trial. Mice administered N-MCQ spent significantly more time in the target quadrant compared to all other treatment groups (p<0.05, group effect). D, swim speed. No differences in swim speed were observed by treatment group. Values represent averages±SEM. *p<0.05, **p<0.01, ***p<0.001.5, p<0.01; Sham×Vehicle, n=9, p<0.05). Values represent averages±SEM. *p<0.05, **p<0.01.

FIG. 11 shows that aminoquinolines decrease microgliosis and astrocytosis in WM tracts of BCAS mice. A, representative Iba-1 and GFAP staining of the medial CC (bregma=0 mm) at day three. B, Iba-1 immuno-positive cell density in multiple WM tracts at day three and 32. Vehicle controls (n=9) had a significantly higher density of Iba-1 positive cells in multiple WM tracts at day three and 32 compared to other treatment groups (n=5). C, GFAP immuno-positive cell density. Vehicle controls had a significantly higher density of GFAP positive cells in the CC at day three and 32 compared to all other treatment groups. Values represent averages±SEM. *p<0.05, **p<0.01, ***p<0.001. positive cells in the CC at day three and 32 compared to all other treatment groups. Values represent averages±SEM. *p<0.05, **p<0.01, ***p<0.001.

FIG. 12 shows that inhibition of QR2 decreases oxidative stress in WM tracts of BCAS mice. The vehicle group (n=9) exhibited significantly higher 8-OhdG staining density than all other treatment groups (CQ, n=8; N-MCQ, n=7; Vehicle, n=9) on day 32 in the IC, and a higher density than sham and N-MCQ groups in the CC on day three. Values represent averages SEM. *p<0.05, **p<0.01, ***p<0.001.

### DETAILED DESCRIPTION

Provided herein are methods of treatment for acute neural injury, vascular dementia or CNS lupus. In some embodiments, quinoline and quinazoline derivatives useful in inhibiting quinone reductase 2 (QR2) are provided for such treatment.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise. As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” and “/” refer to and encompass any and all possible combinations of one or more of the associated listed items, as well as the lack of combinations when interpreted in the alternative (“or”).

### I. Definitions

The following definitions are used herein.

As known in the art, “H” refers to a hydrogen atom. “C” refers to a carbon atom. “N” refers to a nitrogen atom. “O” refers to an oxygen atom.

“Halo” refers to F, Cl, Br or I. “Cl” is chloro, “I” is iodo, “F” is fluoro, and “Br” is bromo.

An “acyl” is a group —C(O)R, where R is a suitable substituent (for example, an acetyl group, a propionyl group, a butyroyl group, a benzoyl group, or an alkylbenzoyl group).

“Alkyl,” as used herein, refers to a straight or branched chain saturated hydrocarbon containing from 1 or 2 to 10 or 20 or more carbon atoms (e.g., C2, C3, C4, C5, C6, C7, C8, C9, C10, C11, C12, C13, C14, C15, etc.). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. In some embodiments, the alkyl is a “lower alkyl” having from 1 to 3, 4, or 5 carbon atoms.

“Alkenyl” as used herein is a straight or branched chain unsaturated hydrocarbon group having one or more double bonds.

“Alkynyl” as used herein is a straight or branched chain unsaturated hydrocarbon group having one or more triple bonds.

“Amino” is the group —NH₂. An “amide” or “amido” as used herein refers to an organic functional group having a carbonyl group (C═O) linked to a nitrogen atom (N). “Alkylamino” refers to an alkyl group, as defined herein, appended to the parent molecule through a nitrogen atom (—NH—).

“Alkoxy,” as used herein, refers to an alkyl group, as defined herein, appended to the parent molecule through an oxygen atom (—O—). Representative examples of alkoxy include, but are not limited to, methoxy, ethoxy, propoxy, 2-propoxy, butoxy, tert-butoxy, pentyloxy, hexyloxy and the like.

“Aryl” as used herein refers to a ring system having one or more aromatic rings. Representative examples of aryl include azulenyl, indanyl, indenyl, naphthyl, phenyl, tetrahydronaphthyl, and the like. The aryl groups of this invention can be substituted with 1, 2, 3, 4, or 5 substituents independently selected from alkenyl, alkenyloxy, alkoxy, alkoxyalkoxy, alkoxycarbonyl, alkyl, alkylcarbonyl, alkylcarbonyloxy, alkylsulfinyl, alkylsulfonyl, alkylthio, alkynyl, aryl, aryloxy, azido, arylalkoxy, arylalkyl, aryloxy, carboxy, cyano, formyl, halo, haloalkyl, haloalkoxy, hydroxy, hydroxyalkyl, mercapto, nitro, sulfamyl, sulfo, sulfonate, NR′R″ (wherein, R′ and R″ are independently selected from hydrogen, alkyl, alkylcarbonyl, aryl, arylalkyl and formyl), and —C(O)NR′R″ (wherein R′ and R″ are independently selected from hydrogen, alkyl, alkylcarbonyl, aryl, arylalkyl, and formyl).

“Cycloalkyl” refers to a monocyclic or fused polycyclic C3 to C10 saturated hydrocarbon groups. “Heterocycloalkyl” refers to a cycloalkyl group in which one or more carbon atoms have been replaced with atoms independently selected from the group consisting of: 0, N, and S.

“Haloalkyl,” as used herein, a refers to a straight or branched chain hydrocarbon containing from 1 or 2 to 10 or 20 or more carbon atoms (e.g., C2, C3, C4, C5, C6, C7, C8, C9, C10, C11, C12, C13, C14, C15, etc.) in which at least one of the hydrogen atoms have been replaced with halo (F, Cl, Br or I). Representative examples of “haloalkyl” include, but are not limited to, fluoroalkyl (e.g., fluoromethyl (—CH₂F), difluoromethyl (—CHF₂), or trifluoromethyl (—CF₃)).

“Heterocyclo,” as used herein, refers to a monocyclic, bicyclic or tricyclic ring system containing at least one heteroatom selected from O, N, and S. Monocyclic heterocycle ring systems are exemplified by any 5 or 6 member ring containing 1, 2, 3, or 4 heteroatoms independently selected from the group consisting of: O, N, and S. The 5 member ring has from 0 to 2 double bonds, and the 6 member ring has from 0 to 3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, sulfoxide, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, pyranopyridine, quinoline, quinolizine, quinoxaline, quinazoline, tetrahydroisoquinoline, tetrahydroquinoline, thiopyranopyridine, and the like. Examples of nitrogen-containing heterocyclo include, but are not limited to, pyrrolidinyl, piperidinyl, piperazinyl, morpholinyl, etc.

“Heteroaryl” means a cyclic, aromatic hydrocarbon in which one or more carbon atoms have been replaced with atoms independently selected from the group consisting of: O, N, and S. Examples of heteroaryl groups include pyridyl, pyrimidinyl, imidazolyl, thienyl, furyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. Preferred heteroaryl groups are five and six membered rings and contain from one to three heteroatoms independently selected from the group consisting of: O, N, and S. The heteroaryl group, including each heteroatom, can be unsubstituted or substituted with from 1 to 4 suitable substituents, as chemically feasible. For example, the heteroatom S may be substituted with one or two oxo groups, which may be shown as ═O. Examples of nitrogen-containing heteroaryls include, but are not limited to, pyridinyl, pyrimidinyl, pyrazinyl, pyridazinyl, imidazolyl, pyrrolyl, pyrazolyl, thiazolyl, triazolyl, isothiazolyl, indolyl, benzimidazolyl, benzoxazolyl, quinolinyl, isoquinolinyl, quinazolinyl, acridinyl, carbazole, azepinyl, 1,4-diazepinyl, purinyl, pteridinyl, phthalazinyl, etc.

“Hydroxyl” and “hydroxy” refer to the group —OH.

“Nitrile” refers to the group —CN.

“Nitro” refers to the group —NO₂.

A “sulfone” refers to a sulfonyl functional group, —SO₂R, wherein R is any covalently linked atom or atoms.

A “sulfoxide” refers to the group —S(O)R, wherein R is any covalently linked atom or atoms.

A “thiol” or “mercapto” refers to the group —SH or to its tautomer=S.

A “ureido” refers to the group —NHCONH₂. A “thioureido” refers to the group —NHCSNH₂.

A “pharmaceutically acceptable salt” is a salt that retains the biological effectiveness of the free acids and bases of a specified compound and that is not biologically or otherwise undesirable. Examples of pharmaceutically acceptable salts include, but are not limited to, sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

A “prodrug” as known in the art is a compound that can be converted under physiological conditions or by solvolysis or metabolically to a specified compound that is pharmaceutically active. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein in their entireties. See also U.S. Pat. No. 6,680,299. Examples include a prodrug that is metabolized in vivo by a subject to an active compound as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. Nos. 6,680,324 and 6,680,322.

As understood in the art, the term “optionally substituted” indicates that the specified group is either unsubstituted, or substituted by one or more suitable substituents. A “substituent” that is “substituted” is a group which takes the place of a hydrogen atom on the parent organic molecule.

### II. Active Compounds

Provided herein as an active compound according to some embodiments is a compound of Formula I:

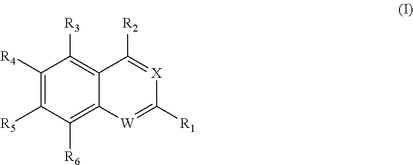

wherein:

W is N or N⁺O⁻;

X is CR₁₄ or N;

R₁ is H or trifluoromethyl;

R₂ is NR₇R₈, OR₁₁, SR₁₂, or alkyl;

R₃ is H or OR₁₃;

R₄ is H or methoxy;

R₅ is H, Cl, or trifluoromethyl;

R₆ is H, NR₉R₁₀ or trifluoromethyl;

R₇ is H, C₁₋₅ alkyl, heteroarylalkyl, cycloalkyl, heterocycloalkyl, heterocyclo, aryl, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo;

R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;

R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;

R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;

R₁ is alkyl, aryl or heteroaryl optionally substituted with alkyl, haloalkyl, aryl or heteroaryl;

R₁₂ is alkyl, aryl or heteroaryl optionally substituted with alkyl, haloalkyl, aryl or heteroaryl;

R₁₃ is alkyl or aryl optionally substituted with alkyl or haloalkyl; and

R₁₄ is H or aryl;

or a pharmaceutically acceptable salt or prodrug thereof.

In some embodiments of Formula I, W is N. In some embodiments of Formula I, X is CR₁₄. In some embodiments of Formula I, R₁ is H. In some embodiments of Formula I, R₂ is NR₇R₈. In some embodiments of Formula I, R₃ is H. In some embodiments of Formula I, R₄ is H. In some embodiments of Formula I, R₅ is Cl. In some embodiments of Formula I, R₆ is H. In some embodiments of Formula I, R₇ is H. In some embodiments of Formula I, R₈ is C₁₋₅ alkyl substituted with heteroaryl. In some embodiments of Formula I, R₁₄ is H.

In some embodiments of Formula I, the compound is a compound of Formula I(a):

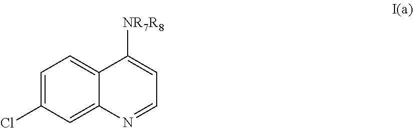

wherein R₇ and R₈ are each independently H or C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments of Formula I(a), one of R₇ and R₈ is hydrogen, and the other is C₁₋₅ alkyl, wherein said C₁₋₅ alkyl is optionally substituted with cycloalkyl, heterocycloalkyl, heterocyclo, aryl, or heteroaryl (which may each be further substituted with any suitable substituent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

In some embodiments of Formula I, the compound is:

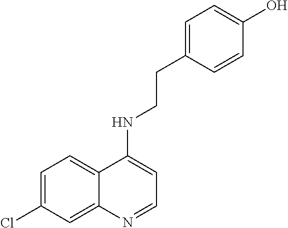 [SMILES: Oc1ccc(CCNc2ccnc3cc(Cl)ccc23)cc1]

4-{2-[(7-chloroquinolin-4-yl)amino]ethyl}phenol

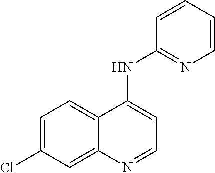 [SMILES: Clc1ccc2c(Nc3ccccn3)ccnc2c1]

7-chloro-N-(pyridin-2-yl)quinolin-4-amine

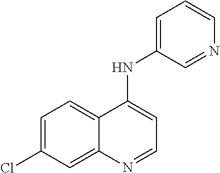 [SMILES: Clc1ccc2c(Nc3cccnc3)ccnc2c1]

7-chloro-N-(pyridin-3-yl)quinolin-4-amine or

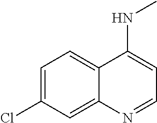

7-chloro-N-methylquinolin-4-amine,

or a pharmaceutically acceptable salt thereof.

In some embodiments of Formula I, the compound is:

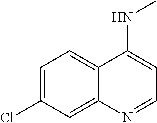

7-chloro-N-methylquinolin-4-amine,

or a pharmaceutically acceptable salt thereof.

Further provided herein as an active compound is a compound of Formula II:

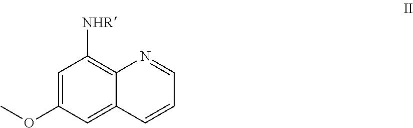

wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,

or a pharmaceutically acceptable salt or prodrug thereof.

In some embodiments of Formula II, the compound is:

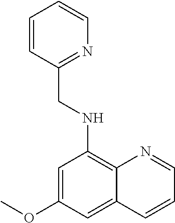 [SMILES: COc1cc(NCc2ccccn2)c2ncccc2c1]

6-methoxy-N-(pyridin-2-ylmethyl)quinolin-8-amine, or

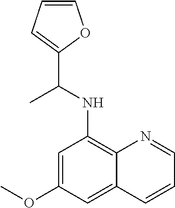 [SMILES: COc1cc(NC(C)c2ccco2)c2ncccc2c1]

N-[1-(furan-2-yl)ethyl]-6-methoxyquinolin-8-amine,

or a pharmaceutically acceptable salt thereof.

Further active compounds may be found in U.S. Patent Application Publication No. 2006/0074105 to Ware, Jr., et al., which application is incorporated by reference herein. The compounds may be prepared according to known methods such as those described in Egan et al., J Med. Chem. 2000, 43:283-291; Stocks et al., J Med. Chem. 2002, 45:4975-4983; or by methods described herein in the examples provided below.

In some embodiments of the above compound of Formula I or Formula II, the compound has a positive log D value at approximately pH 4 to pH 5.

Unless otherwise stated, structures depicted herein are also meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Tautomeric forms include keto-enol tautomers of a compound. In addition, unless otherwise stated, all rotamer forms of the compounds of the invention are within the scope of the invention. Unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

### III. Methods of Use

As noted above, active compounds as taught herein may be useful for treatment of acute neural injury, vascular dementia or CNS lupus.

Acute neural injury includes, but is not limited to, traumatic brain injury and non-traumatic acute brain injury. Traumatic brain injury, as known in the art, is damage and/or dysfunction of the brain caused by a single or repetitive external mechanical force, such as blunt force or sheer force from sudden acceleration or deceleration. Traumatic brain injury includes, but is not limited to, concussion, contusion, and hemorrhage, including parenchymal, subdural, epidural, and subarachnoid hemorrhage. Other acute neural injuries include insult from hypoxic or ischemic brain injury, e.g., from arterial stroke (focal, global), venous infarction, infection, perioperative cerebral injury, etc.

Vascular dementia is dementia or cognitive deficit caused by acute cerebrovascular compromise, often associated with multiple cerebrovascular events such as strokes.

Central nervous system lupus (CNS lupus) refers to neurological and/or behavioral clinical syndromes in subjects with systemic lupus erythematosus (SLE). CNS lupus may present clinically as acute confusion, fatigue, headache, subtle cognitive impairment, delirium, coma, dementia, sensory/motor/autonomic deficits, and/or seizures (the latter which occur more frequently in lupus patients than the general population). CNS lupus may also present as psychological disorders such as depression, mania, and/or psychosis. More focal neurological deficits are also possible and may occur secondary to lupus-related embolic, thrombotic or vasculitic infarction of brain and spine as well as cranial neuropathies. Pathophysiological mechanisms of CNS lupus may include cerebritis, transverse myelitis, neuritis and stroke (embolic, thrombotic, or vasculitic) of the brain or spine.

The term “treat” as used herein refers to any type of treatment that imparts a benefit to a subject afflicted with or at risk of an injury, disease or disorder (e.g., improvement or decreased risk of developing one or more symptoms such as cognitive dysfunction and/or motor dysfunction), delay in the progression of the injury or symptoms, etc.

The present invention is primarily concerned with the treatment of human subjects, but the invention may also be carried out on animal subjects, particularly mammalian subjects such as mice, rats, dogs, cats, livestock and horses for veterinary purposes, and/or for drug screening and/or drug development purposes.

### IV. Formulations

In some embodiments, active compound(s) may be provided in a pharmaceutically acceptable carrier. Carriers should be acceptable in that they are compatible with any other ingredients of the formulation and not harmful to the recipient thereof. In some embodiments, the pharmaceutically acceptable carrier is a sterile (e.g., endotoxin-free or pyrogen-free water, or endotoxin-free or pyrogen-free water saline).

Formulations of the present invention may include short-term, rapid-onset, rapid-offset, controlled release, sustained release, delayed release, and pulsatile release formulations, providing the formulations achieve administration of a compound as described herein. See Remington's Pharmaceutical Sciences (18th ed.; Mack Publishing Company, Eaton, Pa., 1990), herein incorporated by reference in its entirety.

Pharmaceutical formulations according to the present invention may be suitable for various modes of delivery, including oral, parenteral (including intravenous, intramuscular, subcutaneous, intradermal, and transdermal), topical (including dermal, buccal, and sublingual), and rectal administration.

Examples of suitable dosage unit forms in accordance with this invention are tablets, capsules, orally administered liquid preparations in suitable liquid vehicles, sterile preparations in suitable liquid vehicles for intramuscular and intravenous administration, suppositories, and sterile dry preparations for the extemporaneous preparation of sterile injectable preparations in a suitable pharmaceutically acceptable carrier. Suitable solid diluents or carriers for the solid oral pharmaceutical dosage unit forms may be selected from the group consisting of lipids, carbohydrates, proteins and mineral solids; for example, starch, sucrose, kaolin, dicalcium phosphate, gelatin, acacia, corn starch, talc, and the like. Capsules, both hard and soft, may be formulated with suitable diluents and excipients; for example, edible oils, talc, calcium carbonate, and the like, and also, calcium stearate. Liquid preparations for oral administration may be prepared in water or aqueous solutions containing suspending agents; for example, sodium carboxymethylcellulose, methylcellulose, acacia, polyvinyl pyrrolidone, polyvinyl alcohol and the like. In some embodiments, preservatives may be included, for example, parabens, chlorobutanol, benzyl alcohol phenol, and the like. See U.S. Pat. No. 4,159,331 to McCall.

The amount of active compound(s) administered for therapeutic treatment may depend on the age, weight, and condition of the patient as determined by a physician. In some embodiments, the administration and/or pharmaceutical dosage unit form may provide from about 0.05 mg to about 100 mg of the active compound(s) per dosage. In some embodiments, active compound(s) are provided an amount of from about 1 microgram per kg to about 1 g per kg of body weight of the recipient, or 10 micrograms to 100 mg per kg, or 0.1 mg to 50 mg per kg of body weight.

The present invention is explained in greater detail in the following non-limiting examples.

### EXAMPLES

### Example 1: Development of Non-Lysosomotropic Aminoquinoline Inhibitors of QR2

Chloroquine and hydroxychloroquine are lysosomotropic drugs that accumulate preferentially in cellular lysosomes. Their structures are:

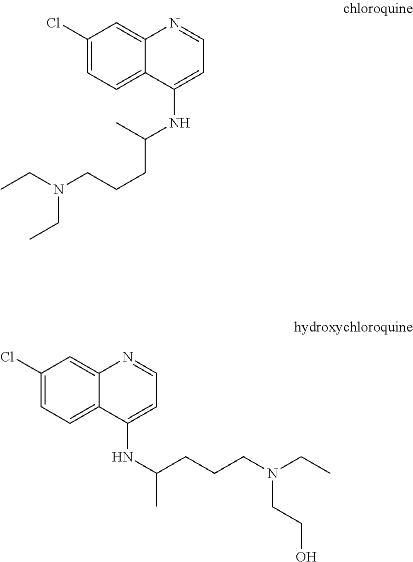 [SMILES: CCN(CC)CCCC(C)Nc1ccnc2cc(Cl)ccc12 ; CCN(CCO)CCCC(C)Nc1ccnc2cc(Cl)ccc12]

For chloroquine, the pKa of the tertiary amine nitrogen is 10.32 and that of the quinoline nitrogen is 7.29. At acidic lysomal pHs between 4 and 5.5, nearly 100% of chloroquine is therefore doubly protonated, rendering the molecule with a 2+ charge that makes the molecule strongly hydrophilic, membrane impermeable, and thus trapped in the acidic organelle.

A quantitative treatment of this trapping phenomenon can be obtained by examining the octanol-water distribution coefficient, log D, of a drug, which depicts the relative partition properties for all forms of a compound at different pH. Compounds with positive log D for a given pH are relatively lipophilic and more membrane permeable, whereas compounds with a negative log D are hydrophilic and less membrane permeable.

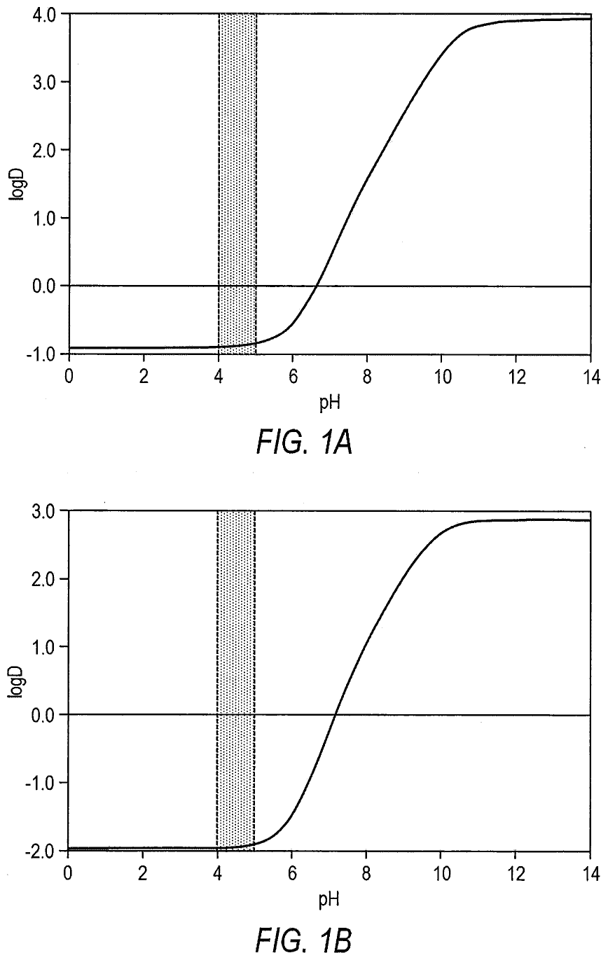

FIG. 1A shows the log D of chloroquine, and FIG. 1B shows the log D of hydroxychloroquine. The shaded regions in the figures represent the potential range of lysosomal pH encountered in vivo. At lysosomal pH, the log D of both chloroquine and hydroxychloroquine are substantially negative, reflecting the accumulated charge of these molecules and their loss of membrane permeability.

A chemoproteomic strategy was used to generate a chemical library of 4-aminoquinoline scaffolds with selectivity for QR2. Using an array of chemi-informatics tools, we have mined this library in silico and have identified aminoquinoline derivatives with nanomolar to micromolar inhibition of QR2 that also possess chemical properties avoiding lysosomal accumulation, thereby addressing the mechanism responsible for CQ/HQ's most common toxicities.

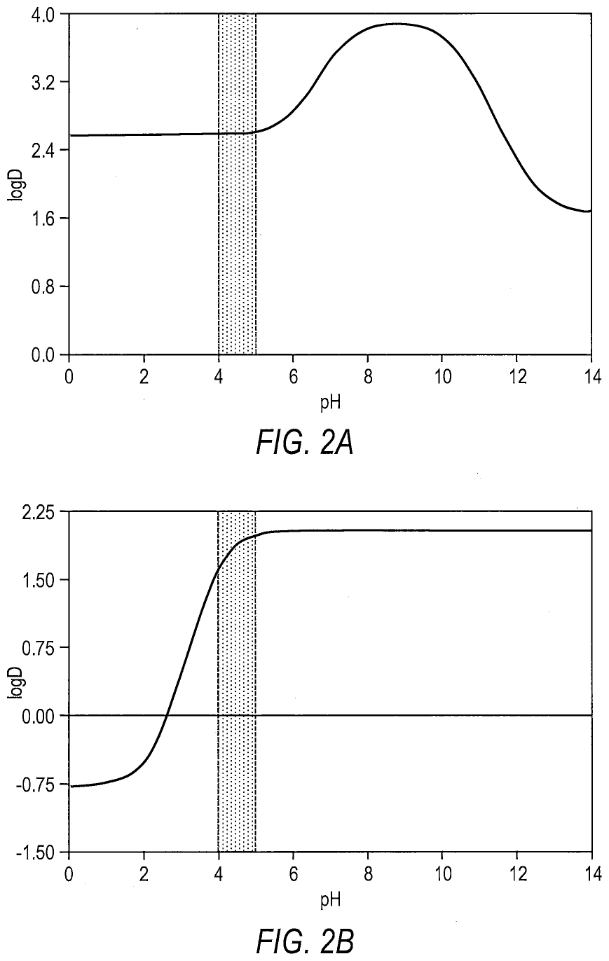

Example compounds as provided below, and their respective log D predictions are shown in FIGS. 2A-2E. Note that regardless of acidity, log D values remain positive (>0.5) for each compound, indicating that these molecules will retain lipophilicity (and thus membrane permeability) at lysosomal pH (between pH 4 and 5).

Example Compound A (log D Shown in FIG. 2A):

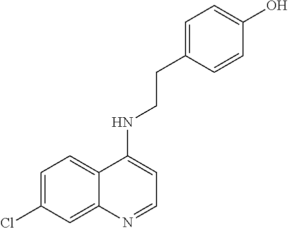 [SMILES: Oc1ccc(CCNc2ccnc3cc(Cl)ccc23)cc1]

4-{2-[(7-chloroquinolin-4-yl)amino]ethyl}phenol

Example Compound B (log D Shown in FIG. 2B):

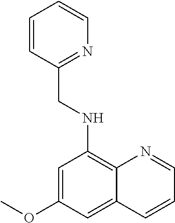 [SMILES: COc1cc(NCc2ccccn2)c2ncccc2c1]

6-methoxy-N-(pyridin-2-ylmethyl)quinolin-8-amine

Example Compound C (log D Shown in FIG. 2C):

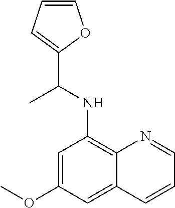 [SMILES: COc1cc(NC(C)c2ccco2)c2ncccc2c1]

N-[1-(furan-2-yl)ethyl]-6-methoxyquinolin-8-amine

Example Compound D (log D Shown in FIG. 2D):

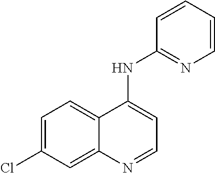 [SMILES: Clc1ccc2c(Nc3ccccn3)ccnc2c1]

7-chloro-N-(pyridin-2-yl)quinolin-4-amine

Example Compound E (log D Shown in FIG. 2E):

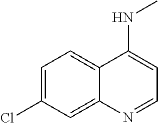

7-chloro-N-methylquinolin-4-amine

Example Compound F:

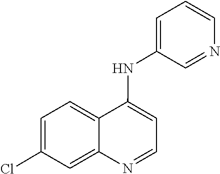 [SMILES: Clc1ccc2c(Nc3cccnc3)ccnc2c1]

7-chloro-N-(pyridin-3-yl)quinolin-4-amine

Table 1 below presents additional estimates of drug-likeness of these non-lysosomotropic 4-aminoquinolines as compared to chloroquine (CQ). Lipophilic efficiency, LiPE (also known as ligand lipophilicity efficiency) is a drug design and discovery parameter linking potency with lipophilicity. LiPE is defined as the pIC₅₀ (−log C₅₀) minus the calculated log P, clog P:

LiPE=pIC₅₀−clogP

LiPE is used to estimate in vivo drug specificity, with higher values predictive of increased potency and decreased probability for unwanted or off-target interactions. LiPE for many of the disclosed 4-aminoquinoline QR2 inhibitors are higher than CQ, therefore predicting a better toxicity profile than CQ has, independent of the substantially reduced toxicity anticipated through elimination of lysosomotropism.

TABLE 1
Empiric and calculated parameters of drug-
likeness for 4-aminoquinoline inhibitors of QR2.

| | | | LiPE |
|---|---|---|---|
| | IC₅₀ (μM) | | (pIC₅₀- |
| Compound | mean +/− SD | clog P | clogP) |
| chloroquine | 1.13 +/− 0.8 | 3.93 | 2.01 |
| 4-{2-[(7-chloroquinolin- | 2.7 +/− 0.2 | 3.92 | 1.65 |
| 4-yl)amino]ethyl}phenol | | | |
| 6-methoxy-N-(pyridin-2- | 0.47 +/− 0.26 | 2.03 | 4.30 |
| ylmethyl)quinolin-8-amine | | | |
| N-[1-(furan-2-yl)ethyl]-6- | 0.85 +/− 0.15 | 2.65 | 3.42 |
| methoxyquinolin-8-amine | | | |
| 7-chloro-N-(pyridin-2- | 0.55 +/− 0.03 | 3.55 | 2.71 |
| yl)quinolin-4-amine | | | |
| 7-chloro-N- | 0.13 +/− 0.03 | 2.21 | 4.67 |
| methylquinolin-4-amine | | | |

### Example 2: 7-Chloro Compound Synthesis and Characterization

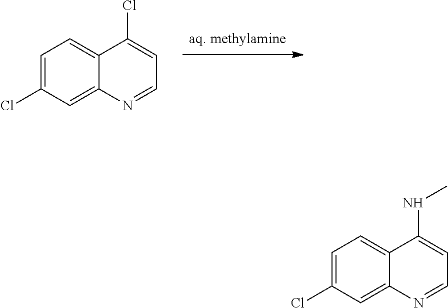

A suspension of 4,7-dichloroquinoline (2.0 g, 10.2 mmol) in aqueous methylamine (40% 20 mL 260 mmol, 26 eq.) was heated in a microwave vessel at 90° C. (initial power setting of 150W) for 2 h. Analysis of the reaction mixture by TLC (2% MeOH in CH₂Cl₂) indicated complete consumption of starting material. The reaction mixture was diluted with H₂O (100 mL) and insoluble were collected at the vacuum. The filter cake was washed with H₂O and dried in vacuo giving the pure product as a white micro crystalline solid (1.8 g, 92%). ¹H NMR (DMSO-d₆, 300 MHz) δ 8.40 (d, J=5.1 Hz, 1H), 8.16 (d, J=9.0 Hz, 1H), 7.77 (s, 1H), 6.38 (d, J=5.4 Hz, 1H), 2.86 (d, J=5.4 Hz, 3H). ESIMS: m/z=193 [(M+H)⁺].

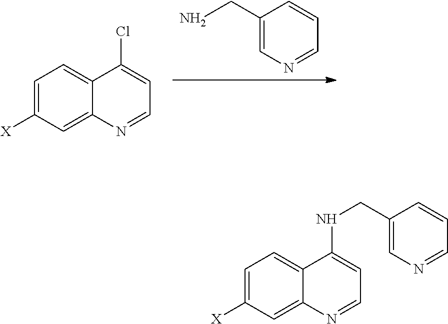

General procedure for 7-substituted-4-(pyridin-3-yl)-methylaminoquinolines. A mixture of the 7-substituted-4-chloroquinoline (5.1 mmol), 3-aminomethyl pyridine (0.70 g, 6.2 mmol, 1.2 eq.) and 1-butanol (5 mL) were heated in a sealed heavy walled pressure vessel (12 mL) at 130° C. (bath temperature) for 24 h. The vessel was cooled to room temperature and the contents were diluted into Et₂O (150 mL). Insolubles were removed at the vacuum. The filter cake was dissolved in a minimum amount of MeOH and the resulting solution was added to silica gel (˜3 g). The mixture was concentrated to dryness under reduced pressure. Flash column chromatography (RediSepR_{f} SiO₂ (40 g), 100% CH₂Cl₂→75% (90:10, CH₂Cl₂:MeOH containing 10% NH₃) gave the desired products.

X═Cl (white solid, 0.92 g, 67%).

¹H NMR (DMSO-d₆, 400 MHz) δ 8.61 (s, 1H), 8.42 (s, 1H), 8.27 (m, 2H), 8.00 (s, 1H), 7.75 (m, 2H), 7.46 (d, J=8.8 Hz, 1H), 7.31 (m, 3H), 6.39 (d, J=5.4 Hz, 1H), 4.55 (d, J=5.4 Hz, 2H). ESIMS: m/z=270 [(M+H)⁺].

### Example 3: Evidence for the Protective Role of QR2 Inhibition in Cerebral Infarction and Therapeutic Effectiveness of Non-Lysosomotropic Inhibitors of QR2

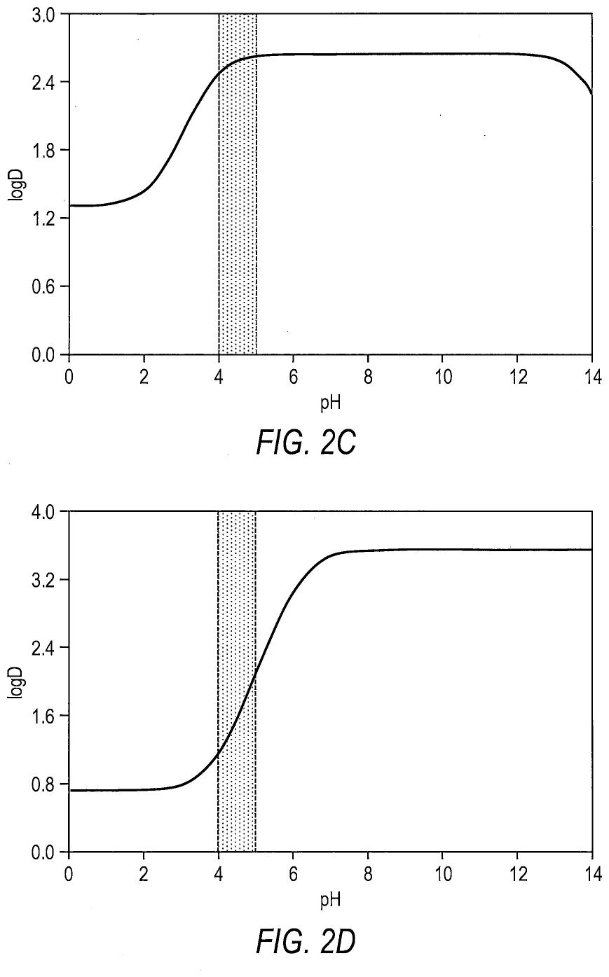

The neuroprotective efficacy of chloroquine (CQ) was demonstrated in a mouse model of transient middle cerebral artery (MCA) occlusion. Post-mortem histological assessments at 72 hours show that a single i.p. administration of CQ (25 mg/kg) 90 minutes after ischemia onset results in a 55% reduction in overall stroke volume, with corresponding reduction in stroke evolution between 4 and 24 hours as measured by diffusion weighted magnetic resonance imaging (DW-MRI), and improvement in neurological score and motor function, as shown in FIGS. 3A-3C.

Also tested was the neuroprotective efficacy of the non-lysosomtropic QR2-selective 4-aminoquinoline, 7-chloro-N-methylquinolin-4-amine (7C-4MAQ, Example Compound F shown above) in the same model. 7-chloro-N-methylquinolin-4-amine results in strikingly significant neuroprotection in this animal model, with reductions in cortical stroke volumes nearly 2× those seen after CQ administration when comparing at an equivalent single, acute phase dose (25 mg/kg) (FIG. 3C).

It is worth noting that the 25 mg/kg dose remains more than 20 times lower than the LD₅₀ determined for this compound. Finally, we compared QR2 null mice to their littermate controls in the same MCA occlusion/reperfusion model, as also shown in FIG. 3C.

### Example 4: Traumatic Brain Injury (TBI)

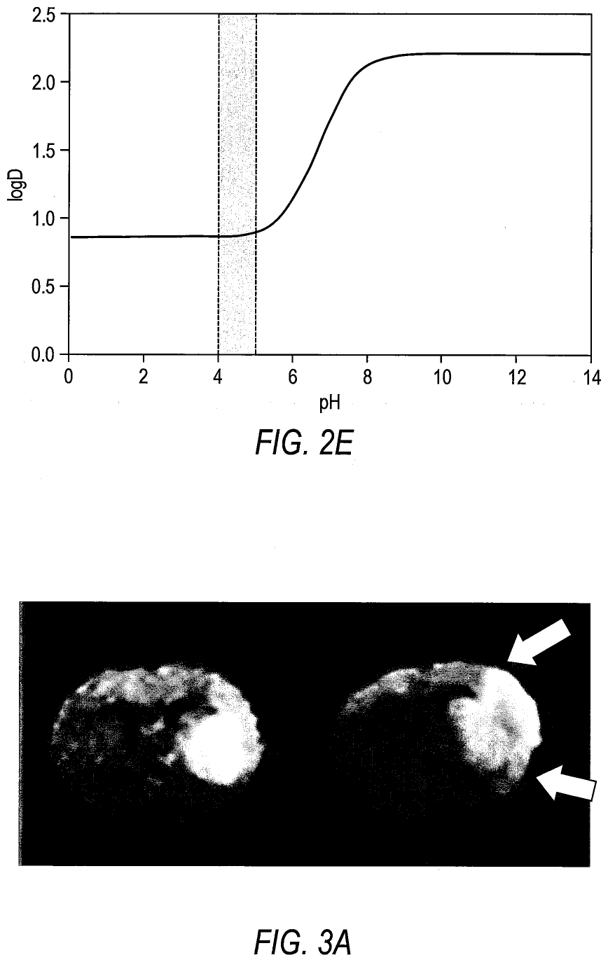

The neuroprotective potential of QR2 inhibition was investigated in a murine TBI model of diffuse closed head injury (Laskowitz et al., “Neuroprotective pentapeptide CN-105 is associated with reduced sterile inflammation and improved functional outcomes in a traumatic brain injury murine model.” Sci. Rep. 2017 Apr. 21; 7:46461). As shown in FIG. 4, marked improvement following TBI was found in both neurocognitive and neuro-motor function assessment. A single 25 mg/kg administration of 7-chloro-N-methylquinolin-4-amine (7C-4MAQ, Example Compound F shown above) resulted in a 20% improvement over vehicle in rotorod latency (n=12/gp), and more significantly, a 62% improvement in Morris water maze performance, even after 1 month following injury (n=12/gp).

As in the stroke model, nearly identical trends are observed when comparing 7C-4MAQ to CQ, and QR2 null mice to their littermate controls.

### Example 5: Intracranial Hemorrhage (ICH)

QR2 inhibition was investigated in a mouse model of intracranial hemorrhagic injury (Lei et al., “Neuroprotective pentapeptide CN-105 improves functional and histological outcomes in a murine model of intracerebral hemorrhage.” Sci. Rep. 2016 Oct. 7; 6:34834). In this study, we investigated 7C-4MAQ, chloroquine (CQ), and an 8-aminoquinonline, primaquine, with the goal of testing the efficacy of the Example Compound while also exploring further the molecular mechanisms of therapeutic action.

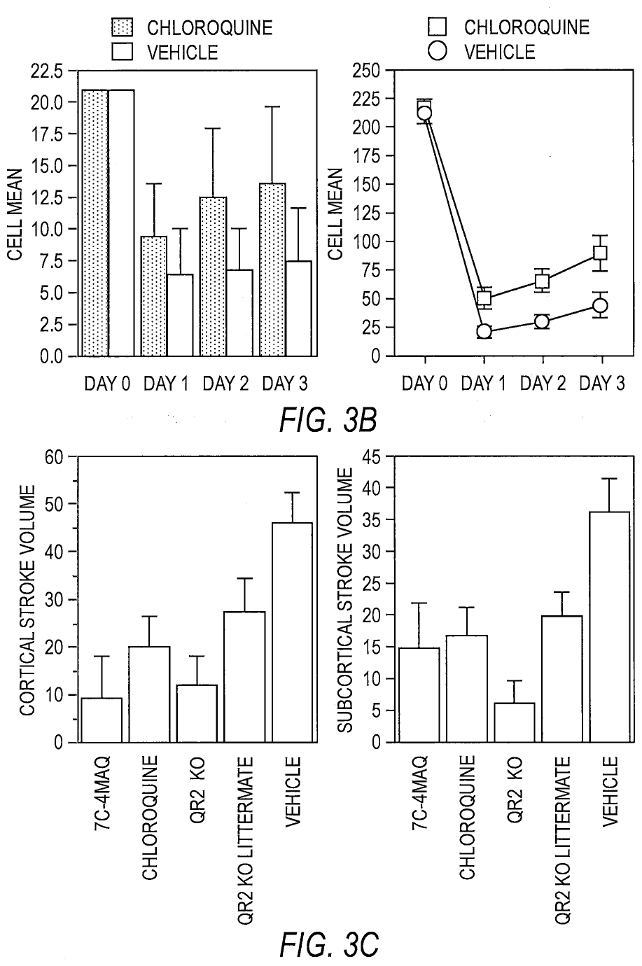

It has been previously reported that aminoquinolines such as CQ and hydroxychloroquine inhibit the second half of the QR2 reaction, whereas other quinolines such as primaquine inhibit the first half. In this particular ICH model (FIG. 5), it is noted that previous neuroprotective interventions have only shown statistically discernable therapeutic efficacy at the histological and molecular but not behavioral level. In our experiments, CQ therapy also resulted in a non-significant 14% (p=0.3) improvement in motor function (rotorod assessment). However, 7C-4MAQ administration resulted in a statistically significant 21% improvement (p=0.013, two-tail t, n=21) in behavioral outcome following a single 25 mg/kg i.p. dose (data not shown). Also noteworthy, primaquine, which selectively inhibits only the first stage of the QR2 reaction, resulted in 35% worsening in motor function (p=0.0001) after an equivalent single i.p. dose (data not shown).

### Example 6: Post-Operative Cognitive Deficit

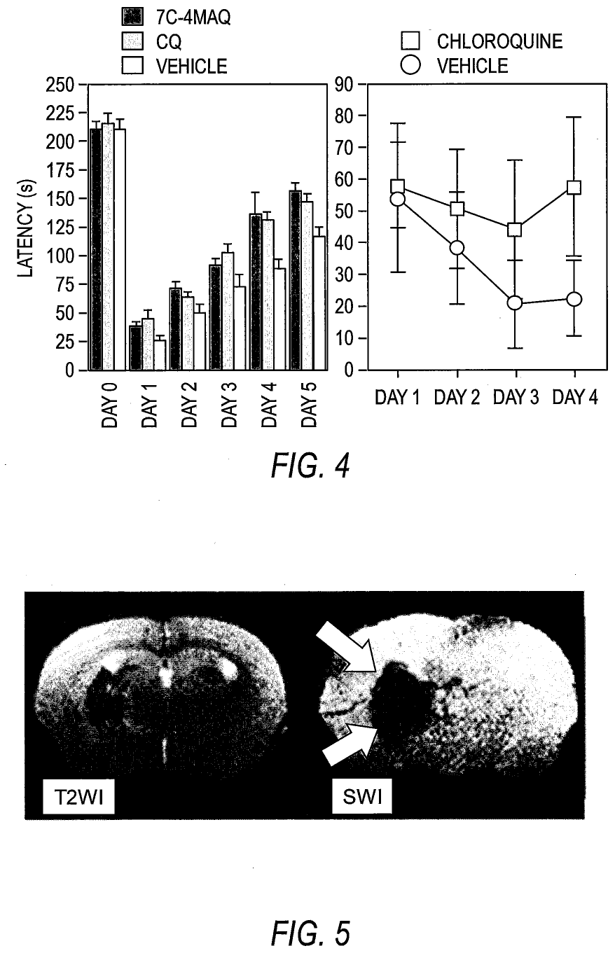

Perioperative cerebral injury (PCI) following major cardiovascular surgery using cardiopulmonary bypass (CPB) and deep hypothermic circulatory arrest (DHCA) remains a significant cause of adverse cerebral outcome. We compared the effects of CQ versus 7C-4MAQ QR2 inhibition on cerebral outcome following cardiopulmonary bypass (CPB)/deep hypothermic circulatory arrest (DHCA) in a well-established rat model originally developed in Dr. Podgoreanu's laboratory at Duke (de Lange et al., “A novel survival model of cardioplegic arrest and cardiopulmonary bypass in rats: a methodology paper,” J Cardiothorac Surg. 2008 Aug. 19; 3:51). Results are shown in FIGS. 6, 7 and 8. For this model of CPB/DHCA, fasting adult male Sprague-Dawley rats (10-12 weeks old) were anaesthetized with inhaled isoflurane 2-2.5%, intubated and mechanically ventilated. Cannulas were placed in the tail artery and the right external jugular vein. Animals were then cooled on CPB for 30 minutes, and DHCA was instituted at a pericranial temperature of 16-18° C. Following 60 minutes of DHCA, CPB was reinitiated, animals were rewarmed for 30 minutes, and separated from CPB at a temperature ≥35.5° C. MRI was performed on day-1 post-operatively, neurological assessments on day 1 and 2 post-operatively, and animals then sacrificed after day 2.

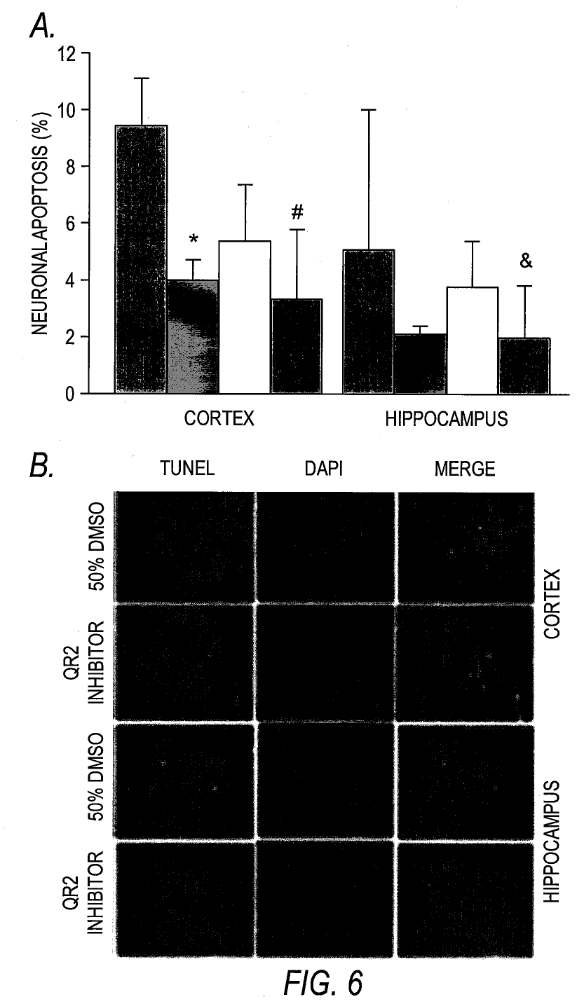

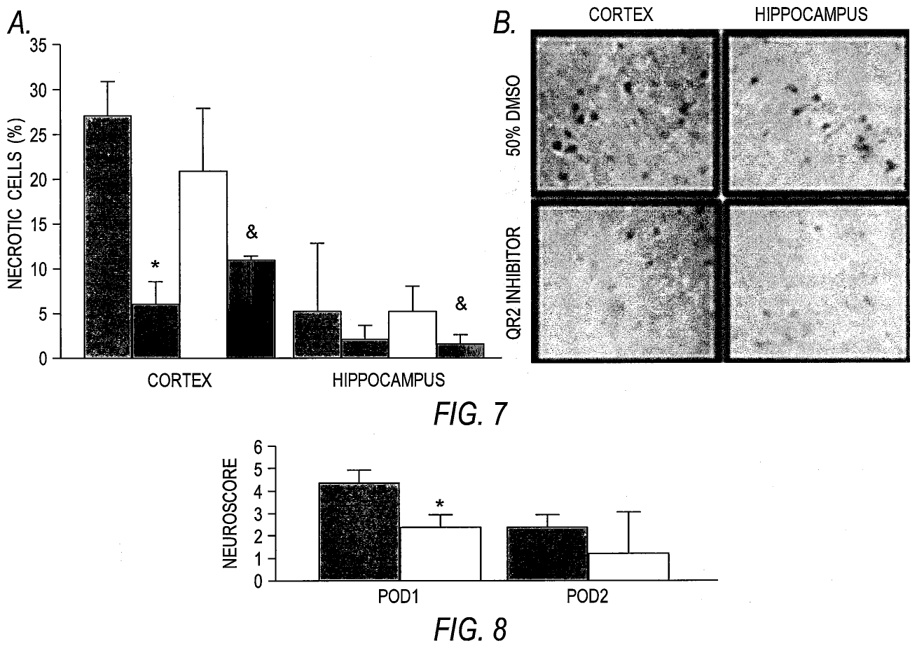

MRI analysis of preliminary results reveals a 3% decrease in post-operative blood brain barrier permeability as measured by gadolinium chelate in animals treated with chloroquine (CQ) or the Example Compound (7C-4MAQ) compared to their respective control groups (p<0.05). Animals treated with either CQ or 7C-4MAQ also show fewer apoptotic and necrotic neurons in cortex and hippocampus (FIG. 6, FIG. 7). Finally, 7C-4MAQ-treated rats demonstrate significantly improved neurological scores at post-op day 1 and 2 (FIG. 8).

### Example 7: Dementia, Vascular Subtype

Vascular dementia is caused by chronic cerebral hypoperfusion and is characterized clinically by white matter lesions on MRI and a decline in executive function. Recent studies have shown that hippocampal expression of quinone oxidoreductase 2 (QR2) is significantly increased in rat models as well as human patients with dementia, suggesting QR2 as a possible therapeutic target. We examined the neuroprotective action of chloroquine and 7C-4MAQ in a murine model of vascular dementia. Physiological, cellular, and functional outcomes were determined using a combination of quantitative immunochemistry, MRI, and behavioral tasks including Morris water maze and rotorod.

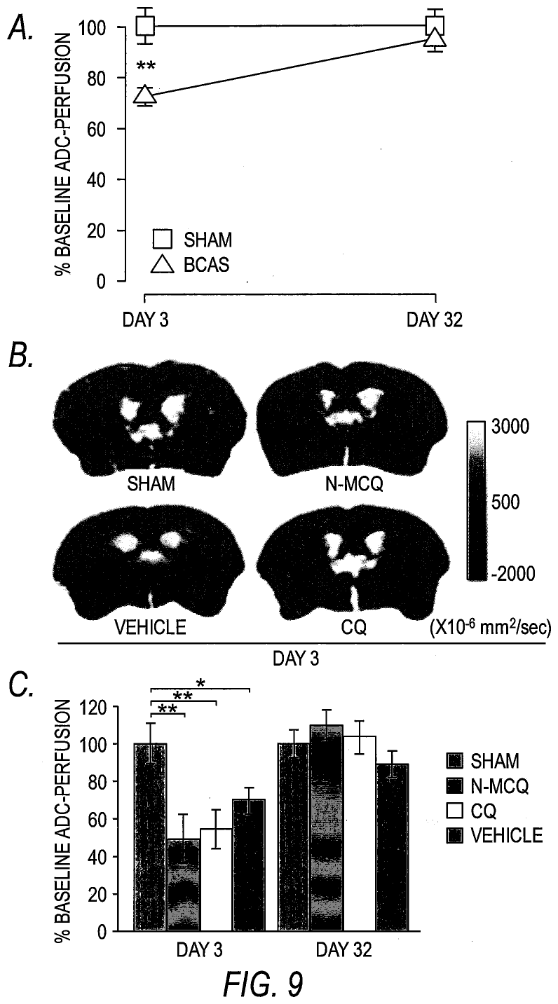

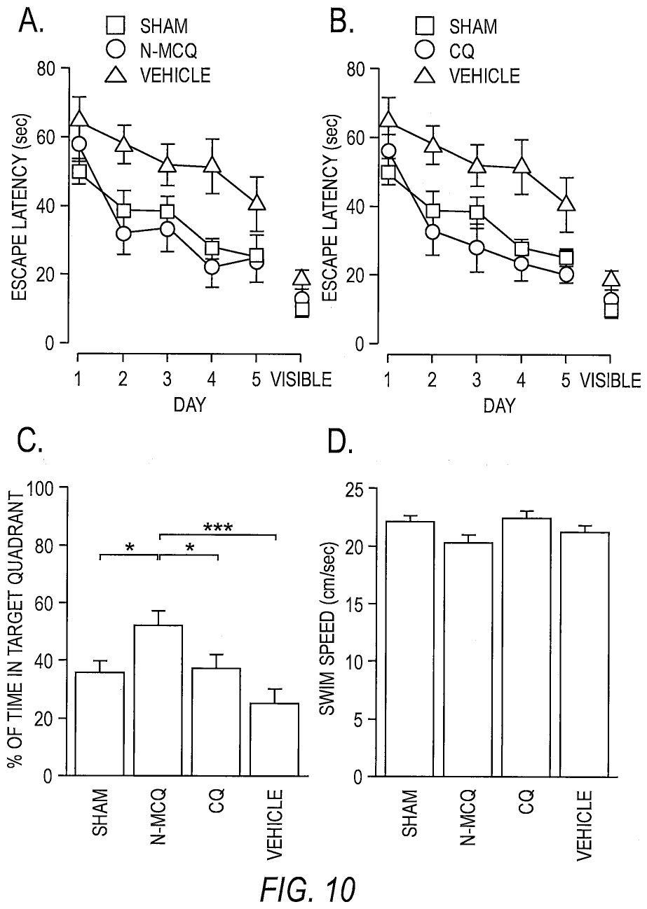

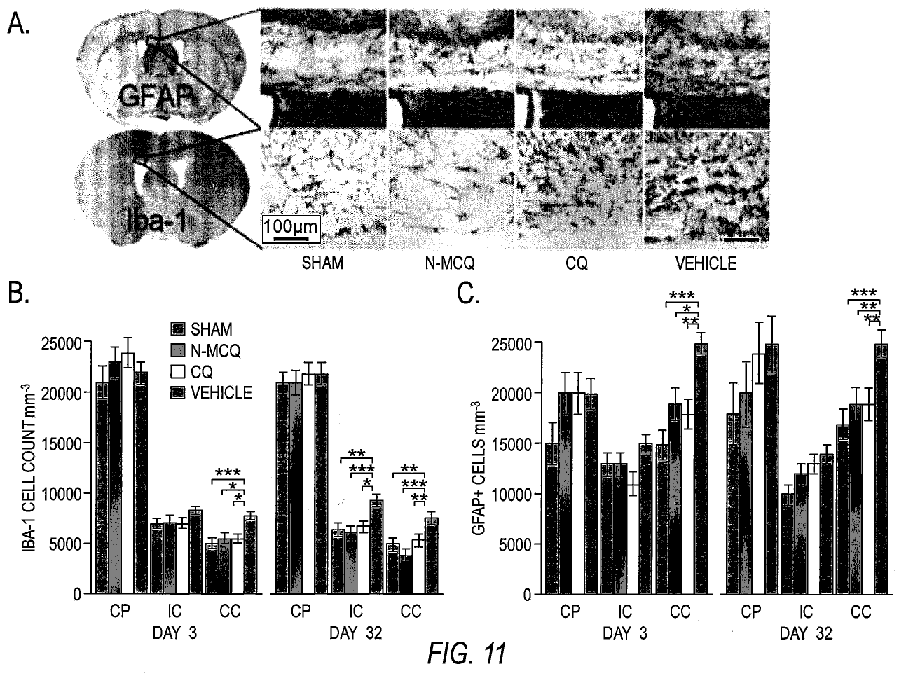

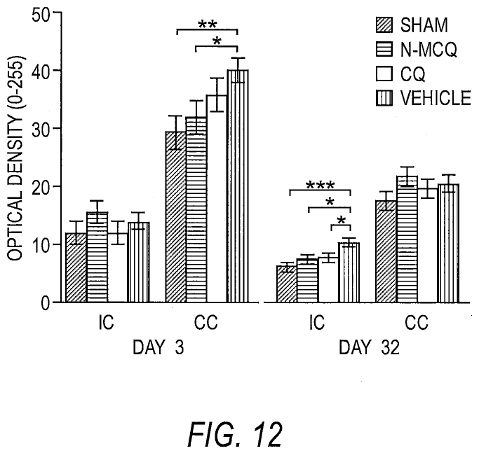

As shown in FIG. 9-FIG. 12, both QR2 inhibitors improved performance on Morris water maze while decreasing astrocytosis, microgliosis, and markers of oxidative stress. Note that in FIG. 9-FIG. 12, 7C-4MAQ is referred to using a previous designation, “N-MCG.” Despite improvements in functional outcome and cellular inflammatory responses, structural markers of white matter injury were unchanged between treatment and control groups. These results provide evidence of a pathologic role for QR2 in dementia and its potential as a therapeutic target. In addition, the results suggest that functionally relevant neuroprotection occurs through mechanisms independent of those responsible for dementia-associated white matter lesions often characterized on MRI.

The foregoing is illustrative of the present invention, and is not to be construed as limiting thereof. The invention is defined by the following claims, with equivalents of the claims to be included therein.

1. A method of treating acute neural injury in a subject in need thereof, comprising administering to the subject a compound of Formula I: 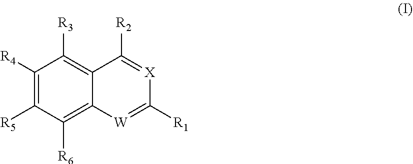
wherein:
W is N or N⁺O⁻;
X is CR₁₄ or N;
R₁ is H or trifluoromethyl;
R₂ is NR₇R₈, OR₁₁, SR₁₂, or alkyl;
R₃ is H or OR₁₃;
R₄ is H or methoxy;
R₅ is H, Cl, or trifluoromethyl;
R₆ is H, NR₉R₁₀ or trifluoromethyl;
R₇ is H, C₁₋₅ alkyl, heteroarylalkyl, cycloalkyl, heterocycloalkyl, heterocyclo, aryl, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo;
R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;
R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;
R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;
R₁₁ is alkyl, aryl or heteroaryl optionally substituted with alkyl, haloalkyl, aryl or heteroaryl;
R₁₂ is alkyl, aryl or heteroaryl optionally substituted with alkyl, haloalkyl, aryl or heteroaryl;
R₁₃ is alkyl or aryl optionally substituted with alkyl or haloalkyl; and
R₁₄ is H or aryl;
or a pharmaceutically acceptable salt or prodrug thereof; or
a compound of Formula II: 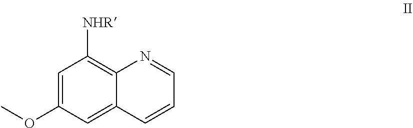
wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,
or a pharmaceutically acceptable salt or prodrug thereof.

2. The method of claim 1, wherein W is N.

3. The method of claim 1, wherein X is CR₁₄ and R₁₄ is H.

4. The method of claim 1, wherein R₂ is NR₇R₈, R₇ is H, and R₈ is C₁₋₅ alkyl substituted with heteroaryl.

5. The method of claim 1, wherein R₁, R₃, R₄, and/or R₆ is H.

6. The method of claim 1, wherein R₅ is Cl.

7. The method of claim 1, wherein said compound is a compound of Formula I(a): 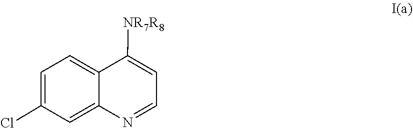
wherein R₇ and R₈ are each independently H or C₁₋₅ alkyl, wherein said C₁₋₅ alkyl is optionally substituted with cycloalkyl, heterocycloalkyl, heterocyclo, aryl, or heteroaryl (which may be further substituted with a suitable substitutent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

8. The method of claim 1, wherein said compound is: 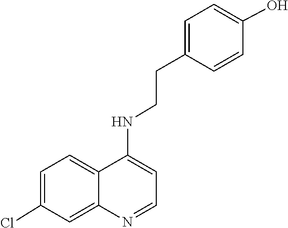 [SMILES: Oc1ccc(CCNc2ccnc3cc(Cl)ccc23)cc1]
4-{2-[(7-chloroquinolin-4-yl)amino]ethyl}phenol, 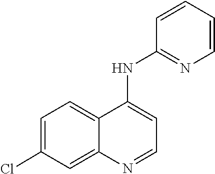 [SMILES: Clc1ccc2c(Nc3ccccn3)ccnc2c1]
7-chloro-N-(pyridin-2-yl)quinolin-4-amine, 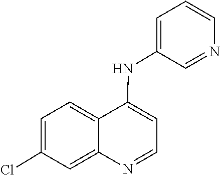 [SMILES: Clc1ccc2c(Nc3cccnc3)ccnc2c1]
7-chloro-N-(pyridin-3-yl)quinolin-4-amine, or 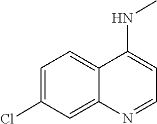
7-chloro-N-methylquinolin-4-amine,
or a pharmaceutically acceptable salt thereof.

9.-10. (canceled)

11. The method of claim 1, wherein said compound is: 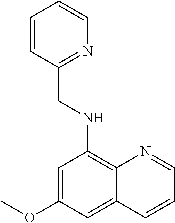 [SMILES: COc1cc(NCc2ccccn2)c2ncccc2c1]
6-methoxy-N-(pyridin-2-ylmethyl)quinolin-8-amine, or 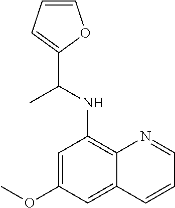 [SMILES: COc1cc(NC(C)c2ccco2)c2ncccc2c1]
N-[1-(furan-2-yl)ethyl]-6-methoxyquinolin-8-amine,
or a pharmaceutically acceptable salt thereof.

12. The method of claim 1, wherein said acute neural injury comprises traumatic brain injury, subarachnoid hemorrhage, post-operative cognitive deficit, hypoxic brain injury, or ischemic brain injury.

13-16. (canceled)

17. A method of treating vascular dementia in a subject in need thereof, comprising administering to the subject a compound of Formula I: 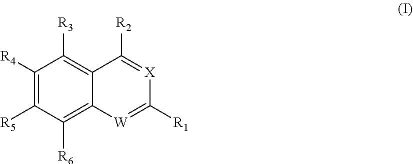
wherein:
W is N or N⁺O⁻;
X is CR₁₄ or N;
R₁ is H or trifluoromethyl;
R₂ is NR₇R₈, OR₁₁, SR₁₂, or alkyl;
R₃ is H or OR₁₃;
R₄ is H or methoxy;
R₅ is H, Cl, or trifluoromethyl;
R₆ is H, NR₉R₁₀ or trifluoromethyl;
R₇ is H, C₁₋₅ alkyl, heteroarylalkyl, cycloalkyl, heterocycloalkyl, aryl, heterocyclo, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo;
R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;
R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;
R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;
R₁₁ is alkyl, aryl or heteroaryl optionally substituted with alkyl, haloalkyl, aryl or heteroaryl;
R₁₂ is alkyl, aryl or heteroaryl optionally substituted with alkyl, haloalkyl, aryl or heteroaryl;
R₁₃ is alkyl or aryl optionally substituted with alkyl or haloalkyl; and
R₁₄ is H or aryl;
or a pharmaceutically acceptable salt or prodrug thereof, or
a compound of Formula II: 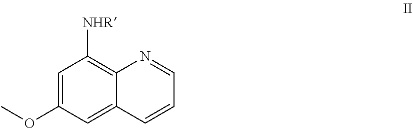
wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,
or a pharmaceutically acceptable salt or prodrug thereof.

18. The method of claim 17, wherein W is N.

19. The method of claim 17, wherein X is CR₁₄ and R₁₄ is H.

20. The method of claim 17, wherein R₂ is NR₇R₈, R₇ is H, and R₈ is C₁₋₅ alkyl substituted with heteroaryl.

21. The method of claim 17, wherein R₁, R₃, R₄, and/or R₆ is H.

22. The method of claim 17, wherein R₅ is Cl.

23. The method of claim 17, wherein said compound is a compound of Formula I(a): 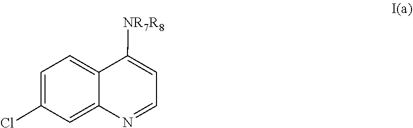
wherein R₇ and R₈ are each independently H or C₁₋₅ alkyl, wherein said C₁₋₅ alkyl is optionally substituted with cycloalkyl, heterocycloalkyl, aryl, or heteroaryl (which may be further substituted with a suitable substitutent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

24. The method of claim 17, wherein said compound is: 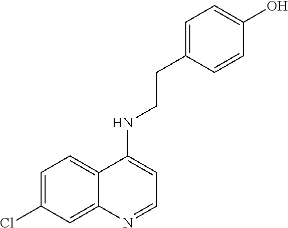 [SMILES: Oc1ccc(CCNc2ccnc3cc(Cl)ccc23)cc1]
4-{2-[(7-chloroquinolin-4-yl)amino]ethyl}phenol, 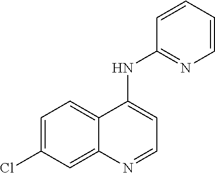 [SMILES: Clc1ccc2c(Nc3ccccn3)ccnc2c1]
7-chloro-N-(pyridin-2-yl)quinolin-4-amine, 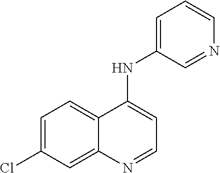 [SMILES: Clc1ccc2c(Nc3cccnc3)ccnc2c1]
7-chloro-N-(pyridin-3-yl)quinolin-4-amine, or 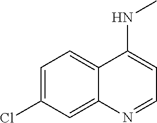
7-chloro-N-methylquinolin-4-amine,
or a pharmaceutically acceptable salt thereof.

25.-26. (canceled)

27. The method of claim 17, wherein said compound is: 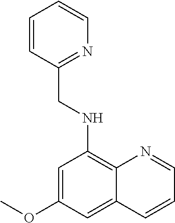 [SMILES: COc1cc(NCc2ccccn2)c2ncccc2c1]
6-methoxy-N-(pyridin-2-ylmethyl)quinolin-8-amine, or 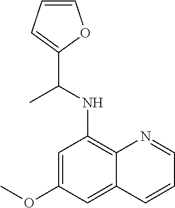 [SMILES: COc1cc(NC(C)c2ccco2)c2ncccc2c1]
N-[1-(furan-2-yl)ethyl]-6-methoxyquinolin-8-amine,
or a pharmaceutically acceptable salt thereof.

28. A method of treating CNS lupus in a subject in need thereof, comprising administering to the subject a compound of Formula I: 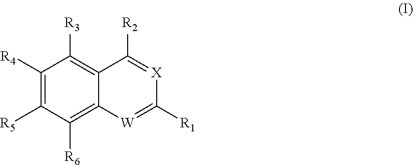
wherein:
W is N or N⁺O⁻;
X is CR₁₄ or N;
R₁ is H or trifluoromethyl;
R₂ is NR₇R₈, OR₁₁, SR₁₂, or alkyl;
R₃ is H or OR₁₃;
R₄ is H or methoxy;
R₅ is H, Cl, or trifluoromethyl;
R₆ is H, NR₉R₁₀ or trifluoromethyl;
R₇ is H, C₁₋₅ alkyl, heteroarylalkyl, cycloalkyl, heterocycloalkyl, heterocyclo, aryl, heteroaryl, ureido, thioureido, alkenyl, alkynyl, amido, amino, alkoxy, alkylamino, alkylphosphonate, alkylnitrile, alkylhalo, or alkylhalo optionally substituted with C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo;
R₈ is H, C₁₋₅ alkyl, cycloalkyl, heterocycloalkyl, aryl, heteroaryl, ureido, thioureido, alkenyl, alkynyl, amido, amino, alkoxy, alkylamino, alkylphosphonate, alkylnitrile, alkylhalo or alkylhalo optionally substituted with C₁₋₅ alkyl, cycloalkyl, cycloalkenyl, heterocycloalkyl, heteroaryl, alkenyl, alkynyl, amido, alkoxy, alkylhydroxy, halo, hydroxyl, carboxylate, alkylcarboxylate, acylazido, sulfonamide or alkylhalo;
R₉ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, aryl, heteroaryl or alkylamino;
R₁₀ is H, O, C₁₋₅ alkyl, cycloalkyl, heterocycloalkyl, aryl, heteroaryl, alkylamino, alkylnitrile or alkylphosphonate optionally substituted with C₁₋₅ alkyl, cycloalkyl, heterocycloalkyl, heteroaryl, or alkylamino;
R₁₁ is alkyl, aryl or heteroaryl optionally substituted with alkyl, haloalkyl, aryl or heteroaryl;
R₁₂ is alkyl, aryl or heteroaryl optionally substituted with alkyl, haloalkyl, aryl or heteroaryl;
R₁₃ is alkyl or aryl optionally substituted with alkyl or haloalkyl; and
R₁₄ is H or aryl;
or a pharmaceutically acceptable salt or prodrug thereof, or
a compound of Formula II: 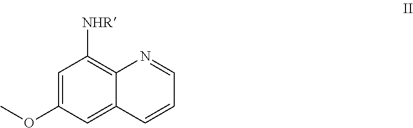
wherein R′ is selected from the group consisting of pyridin-2-ylmethyl, pyridin-3-ylmethyl, 1-benzylpiperidin-4-yl, 4-cyano-2,2-diethylbutyl, 2-chlorocyclopentyl, 4-(diethylamino)butan-2-yl, 1-(furan-2-yl)ethyl, 1-cyclopropylethyl, 1-ethylpiperidin-4-yl, 5-amino-2,2-diethylpentyl, and 2-(diethylphosphoryl)-1-methylethyl,
or a pharmaceutically acceptable salt or prodrug thereof.

29. The method of claim 28, wherein W is N.

30. The method of claim 28, wherein X is CR₁₄ and R₁₄ is H.

31. The method of claim 28, wherein R₂ is NR₇R₈, R₇ is H, and R₈ is C₁₋₅ alkyl substituted with heteroaryl.

32. The method of claim 28, wherein R₁, R₃, R₄, and/or R₆ is H.

33. The method of claim 28, wherein R₅ is Cl.

34. The method of claim 28, wherein said compound is a compound of Formula I(a): 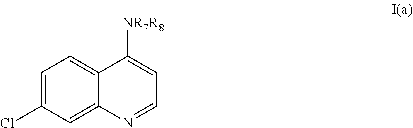
wherein R₇ and R₈ are each independently H or C₁₋₅ alkyl, wherein said C₁₋₅ alkyl is optionally substituted with cycloalkyl, heterocycloalkyl, aryl, or heteroaryl (which may be further substituted with a suitable substitutent, e.g., C₁₋₅ alkyl, cycloalkyl, cycloalkenyl, heterocycloalkyl, heteroaryl, alkenyl, alkynyl, amido, alkoxy, alkylamino, alkylhydroxy, halo, hydroxyl, carboxylate, alkylcarboxylate, acylazido, sulfonamide or alkyl halo).

35. The method of claim 28, wherein said compound is: 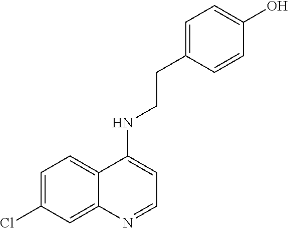 [SMILES: Oc1ccc(CCNc2ccnc3cc(Cl)ccc23)cc1]
4-{2-[(7-chloroquinolin-4-yl)amino]ethyl}phenol, 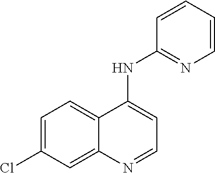 [SMILES: Clc1ccc2c(Nc3ccccn3)ccnc2c1]
7-chloro-N-(pyridin-2-yl)quinolin-4-amine, 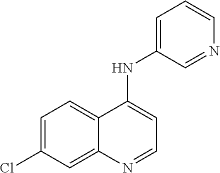 [SMILES: Clc1ccc2c(Nc3cccnc3)ccnc2c1]
7-chloro-N-(pyridin-3-yl)quinolin-4-amine, or 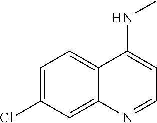
7-chloro-N-methylquinolin-4-amine,
or a pharmaceutically acceptable salt thereof.

36.-37. (canceled)

38. The method of claim 28, wherein said compound is: 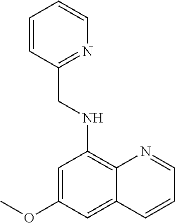 [SMILES: COc1cc(NCc2ccccn2)c2ncccc2c1]
6-methoxy-N-(pyridin-2-ylmethyl)quinolin-8-amine, or 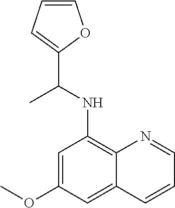 [SMILES: COc1cc(NC(C)c2ccco2)c2ncccc2c1]
N-[1-(furan-2-yl)ethyl]-6-methoxyquinolin-8-amine,
or a pharmaceutically acceptable salt thereof.

39. The method of claim 1, wherein said compound has a positive log D value at approximately pH 4 to pH 5.
